(19) United States
(12) Patent Application Publication
(10) Pub. No.: US 2025/0152963 A1
(43) Pub. Date: May 15, 2025

(54) THERAPEUTIC LIGHT EFFECTUATING PHYSIOLOGICAL RESPONSES

(71) Applicant: Haven Technologies Inc., Alexandra, VA (US)

(72) Inventors: Rami Burstein, Chestnut Hill, MA (US); Fredric Maxik, W. Warwick, RI (US)

(73) Assignee: Have Technologies Inc., Alexandra, VA (US)

(21) Appl. No.: 18/839,207

(22) PCT Filed: Feb. 17, 2023
(86) PCT No.: PCT/US2023/062845

§ 371 Date: Aug. 16, 2024

Related U.S. Application Data
(60) Provisional application No. 63/268,147, filed on Feb. 17, 2022.

(51) Int. Cl. A61N 5/06

(52) U.S. Cl. CPC A61N 5/0613; A61N 2005/0626; A61N 2005/0663; A61N 2005/0666

(57) ABSTRACT

A system for selective emission of therapeutic monochromatic light including a light source operable to emit light within a monochromatic wavelength range, defining a therapeutic light, the monochromatic wavelength range being associated with a physiological response in a human subject, a non-transitory computer-readable storage medium, and a controller positioned in communication with each of the non-transitory computer-readable medium and the light source and operable to receive an indication of optical characteristics of a space into which the light source is positioned to emit light, defined as a spatial optical characteristic and operate the light source to emit the therapeutic light responsive to the spatial optical characteristic.

### FIELD OF THE INVENTION

The present invention relates to systems and methods for emitting therapeutic light within a wavelength range responsive to optical characteristics of the space into which the light is emitted.

### BACKGROUND OF THE INVENTION

The impact of lighting on one or more biological rhythms is of increasing relevance and concern. Solutions directed towards controlling the emission of light to control the effect of emitted light on biological rhythms, described herein as therapeutic lighting, either promoting or selectively hindering such rhythms, is being increasingly implemented. For example, the impact of light within the blue visible spectrum and its affect on circadian rhythms of those observing the blue light, including light emitted by personal electronics, is of increasing concern. However, systems and apparatuses for selectively affecting biological rhythms have been limited to considering only the spectral power distribution of the light as it is emitted from the device and directly irradiating the observer. Such solutions fail to consider characteristics of the environment into which the light is emitting. Environments and vary widely on a multitude of optical characteristics, including, but not limited to, reflectivity and the presence of electronic display devices within the space. Such solutions may be emitting therapeutic light at a greater intensity that is necessary, or may not sufficiently be avoiding light within a biologically-effective spectrum when exposure to such light is cumulative with other light sources in the environment. Accordingly, there is a need in the art for a lighting device that can identify such spatial optical characteristics and adjust the characteristics of light emitted thereby responsive thereto.

Additionally, prior solutions have typically emitted therapeutic light into the environment without consideration as to the distance and/or location of a target of the biologically-effective lighting, instead merely emitting light with a uniform spectral power distribution to accomplish the biological effect regardless as to whether such an effect is likely, or indeed possible based on the target being too far to be affected, or altering the intensity of the therapeutic light more than is necessary relative to the proximity of the target to the lighting device when a lower intensity would be sufficiently effective. Accordingly, there is a need in the art for a lighting system that can consider these types of spatial optical characteristics and adjust the characteristics of light emitted thereby.

### SUMMARY OF THE INVENTION

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

One general aspect includes a system for selective exposure to therapeutic monochromatic light. The system also includes a light source operable to emit light within a monochromatic wavelength range, defining a therapeutic light, the monochromatic wavelength range being associated with a physiological response in a human subject. The system also includes a non-transitory computer-readable storage medium. The system also includes a controller positioned in communication with each of the non-transitory computer-readable medium and the light source and configured to: receive an indication of optical characteristics of a space into which the light source is positioned to emit light, defined as a spatial optical characteristic; and operate the light source to emit the therapeutic light responsive to the spatial optical characteristic. Other embodiments of this aspect include corresponding computer systems, apparatus, and computer programs recorded on one or more computer storage devices, each configured to perform the actions of the methods.

Implementations may include one or more of the following features. The system where the monochromatic wavelength range has a half-wave width of not more than 10 nanometers (nm). In alternative embodiments, the have-wave width may be 20 nm. The monochromatic wavelength range may be centered at 520 nm. The controller is configured to operate the light source responsive to the spatial optical characteristics by controlling at least one of a peak intensity, a fluence, and a luminous flux of the therapeutic light responsive to the spatial optical characteristics. The spatial optical characteristic may include at least one of a spectral power distribution of the space, a reflectivity characteristic of the space, a reflectivity model of the space, a distance to a target of the therapeutic light, a location of the target within the space, a location of the target relative to the light source, and a presence of an electronic display device in the space. The spatial optical characteristics may include: a distance to the target, and at least one of a location of the target within the space and a location of the target relative to the light source, and the controller is configured to: calculate a horizontal intensity efficiency of therapeutic light emitted by the light source responsive to the spatial optical characteristics, and operate the light source further responsive to the horizontal intensity efficiency. The spatial optical characteristic is received by the digital communication device and transmitted to the controller. The system may include software operable to run on a smartphone device and configured to: obtain an optical measurement of the space from an optical sensor may include by the smartphone device, and determine the spatial optical characteristic from the optical measurement, and transmit the spatial optical characteristic to the digital communication device utilizing a digital communication device may include by the smartphone device. The controller is configured to: operate the optical sensing device to obtain an optical measurement of the space, defining an obtained optical measurement; and determine the spatial optical characteristic of the space from the obtained optical measurement. The system may include: a housing configured to carry the light source, and an optic configured to at least one of reflect and refract the therapeutic light emitted by the light source into the space. The housing, the optic, and the light source define a troffer light fixture configured to be installed in a ceiling of the space; and the optic is configured to at least one of reflect and refract the therapeutic light such that it is emitted into the space at an angle greater than 45 degrees relative to a normal of the troffer light fixture. Implementations of the described techniques may include hardware, a method or process, or computer software on a computer-accessible medium.

Another general aspect includes a method of administering therapeutic light to a subject to effectuate a physiological response. The method of administering therapeutic light also includes receiving an indication of optical characteristics of a space into which the light source is positioned to emit light, defined as a spatial optical characteristic. The light also includes determining at least one emission characteristic responsive to the spatial optical characteristic to emit light within a monochromatic wavelength range, defining a therapeutic light, the monochromatic wavelength range being associated with a physiological response in a human subject. The light also includes emitting the therapeutic light into the space. Other embodiments of this aspect include corresponding computer systems, apparatus, and computer programs recorded on one or more computer storage devices, each configured to perform the actions of the methods.

Implementations may include one or more of the following features. The method where the monochromatic wavelength range has a half-wave width of not more than 10 nm. The monochromatic wavelength range is centered at 520 nm. The spatial optical characteristic may include at least one of a spectral power distribution of the space, a reflectivity characteristic of the space, a reflectivity model of the space, a distance to a target of the therapeutic light, and a presence of an electronic display device in the space. The spatial optical characteristic is received from at least one of a directly connected computerized device and a computerized device connected across a computerized network. The method may include: obtaining an optical measurement of the space, defining an obtained optical measurement; and determining the spatial optical characteristic of the space from the obtained optical measurement. The method may include providing an indication to a user to alter the spatial optical characteristic, defining an indicated alteration. The indicated alteration to the spatial optical characteristic may include at least one of applying an optically-reflective coating to a surface within the space and positioning an optically-reflective object within the space. The emission characteristic is at least one of a peak intensity, a fluence, and a luminous flux of the therapeutic light. Implementations of the described techniques may include hardware, a method or process, or computer software on a computer-accessible medium.

### BRIEF DESCRIPTION OF THE DRAWINGS

FIG. 1 is a schematic view of a system for selective emission of therapeutic monochromatic light according to an embodiment of the invention.

FIG. 2 is a perspective view of a lighting system implemented in a space according to an embodiment of the invention.

FIG. 3 is a perspective view of a troffer light fixture operable to emit therapeutic light according to an embodiment of the invention.

FIG. 4 is a flowchart illustrating a method according to an embodiment of the invention.

### DETAILED DESCRIPTION OF THE INVENTION

The present invention will now be described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Those of ordinary skill in the art realize that the following descriptions of the embodiments of the present invention are illustrative and are not intended to be limiting in any way. Other embodiments of the present invention will readily suggest themselves to such skilled persons having the benefit of this disclosure. Like numbers refer to like elements throughout.

Although the following detailed description contains many specifics for the purposes of illustration, anyone of ordinary skill in the art will appreciate that many variations and alterations to the following details are within the scope of the invention. Accordingly, the following embodiments of the invention are set forth without any loss of generality to, and without imposing limitations upon, the invention.

In this detailed description of the present invention, a person skilled in the art should note that directional terms, such as “above,” “below,” “upper,” “lower,” and other like terms are used for the convenience of the reader in reference to the drawings. Also, a person skilled in the art should notice this description may contain other terminology to convey position, orientation, and direction without departing from the principles of the present invention.

Furthermore, in this detailed description, a person skilled in the art should note that quantitative qualifying terms such as “generally,” “substantially,” “mostly,” and other terms are used, in general, to mean that the referred to object, characteristic, or quality constitutes a majority of the subject of the reference. The meaning of any of these terms is dependent upon the context within which it is used, and the meaning may be expressly modified.

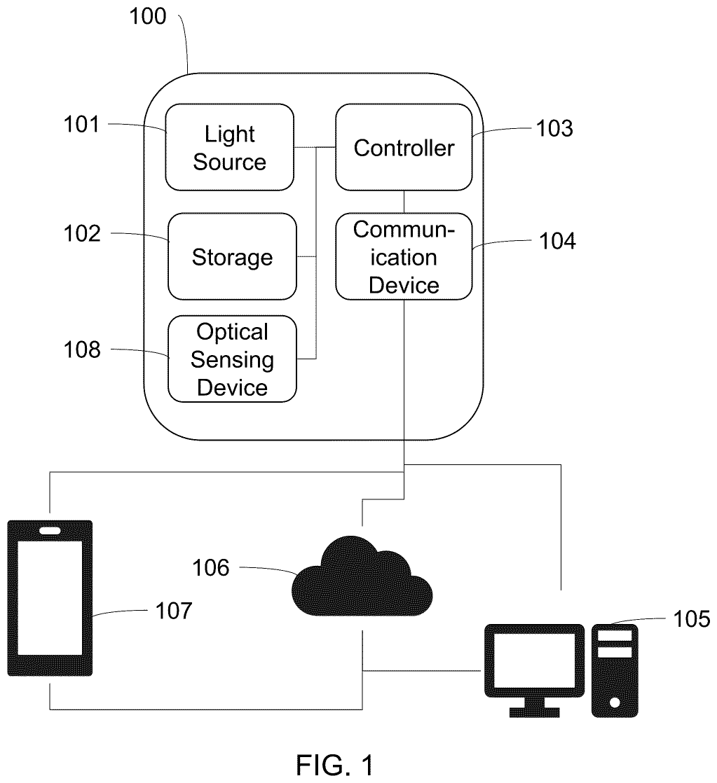

An embodiment of the invention, as shown and described by the various figures and accompanying text, provides a system 100 for selective emission of therapeutic monochromatic light. Referring specifically to FIG. 1, the system 100 may include a light source 101, a non-transitory computer-readable storage medium 102, and a controller 103.

The light emitted by the light source 101 may be within a monochromatic wavelength range and may be referred to as a therapeutic light. The monochromatic wavelength range of the therapeutic light may cause a beneficial physiological response in a human subject when the therapeutic light enters the subject's retinas. In one embodiment, the monochromatic wavelength range may have a half-wave width of not more than 10 nm. In another embodiment, the monochromatic wavelength range may have a half-wave width of not more than 20 nm. The monochromatic wavelength range may be centered at 520 nm. In another embodiment, the monochromatic wavelength range may be centered at a wavelength within a range from 510 nm to 530 nm. A therapeutic light centered at 520 nm and having a half-wave width of not more than 10 nm may be a very narrow wavelength band and be referred to as green light. This very narrow wavelength band of green light may provide particular therapeutic benefits to human subjects when it enters their retinas.

By way of example, and not as a limitation, the benefits to the subjects may include reduced eyestrain, improved concentration, improved sleep quality, improved tolerance to working in front of a screen, and the ability to work (or work longer) in front of a screen.

Eyestrain is a common condition that occurs when a subject's eyes get tired from intense use. Activities that can lead to eyestrain include, but are not limited to, driving long distances and staring at computer screens and other digital devices. Symptoms of eyestrain may include, but are not limited to, sore eyes, tired eyes, burning or itching eyes, watery or dry eyes, blurred or double vision, headache, sore neck, sore shoulders, sore back, increased sensitivity to light, difficulty concentrating, or feeling that one cannot keep his or her eyes open. Exposure to therapeutic light may reduce eyestrain experienced by a subject.

Improved sleep quality can provide many benefits to a subject. Exposure to therapeutic light may improve sleep quality, thereby providing benefits to subjects in a workplace. Improving sleep quality may reduce daytime sleepiness, reduce general fatigue, reduce appetite, and, consequently, reduce the risk of gaining weight unintentionally. Exposure to therapeutic light before bedtime may improve the ability of a subject to fall asleep, allow a subject to fall asleep faster, reduce the number of trips to the bathroom during the night, or reduce the number of episodes of nighttime awakening.

Exposure to therapeutic light may improve concentration, which may allow a subject to complete tasks more quickly, with greater creativity, or while making fewer mistakes. Improved concentration may also result in a subject producing a higher quality work product, not falling behind on his or her tasks, or, consequently, feeling less stressed. These benefits due to improved concentration generally lead to an overall improved performance at work, which can lead to greater career opportunities and advancement, as well as potentially higher income.

Exposure to therapeutic light may improve a subject's tolerance for working in front of a screen, including, but not limited to, computers, tablets, cellular phones, and the like. The benefits of this improved tolerance may include reduced shoulder and back discomfort and a reduced incidence of depression, anxiety, or irritability due to screen usage.

Finally, exposure to therapeutic light may allow a subject to work, or to work for longer hours, in front of a screen. The benefits of an increased ability to work in front of a screen include, but are not limited to, increased opportunities for promotion, increased earning potential, improved company productivity, or potential for earning more days off.

The non-transitory computer-readable storage medium 102 may be in communication with the controller 103 and may store software configured to perform the functions described hereinbelow.

The controller 103 may be in communication with both the non-transitory computer-readable storage medium 102 and the light source 101. In one embodiment, the controller 103 may receive an indication of optical characteristics of a space into which the light source is positioned to emit light. The indications may be referred to as spatial optical characteristics. The controller 103 may adjust one or more emission characteristics of the light source 101 based on one or more spatial optical characteristics. In one embodiment, the emission characteristics of the light source 101 that may be controlled by the controller 103 may include one or more of peak intensity, fluence, and luminous flux. The controller 103 may control these emission characteristics of the light source 101 in any combination. The controller 103 may control these emission characteristics of the light source 101 in response to inputs received by the controller 103, which may be provided by one or more sensors.

In addition to controlling these emission characteristics, the controller may be adapted to dim the light source 101, operate the light source 101 based on a timer, activate the light source 101 only when a subject is present in the space, and operate the light source 101 only when ambient light does not provide adequate lighting for a subject.

The controller 103 may receive information related to the spatial optical characteristics of the space in which the system 100 is positioned. Spatial optical characteristics may include, but are not limited to, spectral power distribution of the space, reflectivity characteristic of the space, reflectivity model of the space, distance to a target of the therapeutic light, location of the target within the space, location of the target relative to the light source, distance to an object in the space, absorption of an object in the space, reflectivity of an object in the space, position of the system 100 within the space, and a presence or absence of an electronic display device in the space.

The system 100 may utilize the spatial optical characteristics of the space to determine the desired emission characteristics of the light source 101. In one embodiment, the controller may receive spatial optical characteristics of the space and calculate desired emission characteristics while taking into account how much therapeutic light is in the field of view of the subject or how much therapeutic light enters the subject's retinas. Such a calculation may be based on the subject's actual or anticipated position in the space, along with other spatial optical characteristics.

The system 100 may include one or more sensors, which may include optical sensors 108. Each optical sensor 108 may be in electrical communication with the controller 103. The optical sensors 108 may detect one or more spatial optical characteristics of the space in which the sensor is physically located. Based on the spatial optical characteristics of the space, the controller 103 may configure the emission characteristics of the light source 101 to optimize the therapeutic light received by a subject located in the space. Any type of optical sensor 108 as is known in the art is contemplated and included within the scope of the invention, including, but not limited to, photodiodes, photoconductive devices, photovoltaics, phototransistors, and optical sensors. With regard to location-sensing functions of the sensors, it is contemplated and included within the scope of the invention that acoustic-sensing devices may be comprised by the system 100, including ultrasonic devices.

The controller 103 may operate the optical sensor 108 or other sensor to obtain an optical measurement of the space in which the optical sensor 108 or other sensor is located and define the obtained optical measurement. The controller 103 may also determine a spatial optical characteristic of the space from the optical measurement obtained by the optical sensor 108 or other sensor and defined by the controller 103. Such spatial optical characteristics may include those mentioned above, as well as measuring light having a spectral power distribution suggesting it is emitted from a computer display, from outside, and the otherwise ambient light within the space. In one embodiment, the controller 103 may calculate an optimized spatial optical characteristic and provide an indication to a user of the system 100 that the space may, or should, be modified to increase the therapeutic effect of the system 100. In some embodiments, where the subject is likely to be looking at a computer display, the optimized spatial optical characteristic may be determined responsive to the likely increased blue light exposure the subject is receiving. The system 100 may provide an indication to a user to alter one or more spatial optical characteristics. Such alterations may result in improved therapeutic performance of the system 100. The indication to the user to alter one or more spatial optical characteristic may be referred to as an indicated alteration. By way of example, and not was a limitation, an indicated alteration may include applying an optically-reflective coating to a surface within the space or positioning an optically-reflective object within the space. Such an indication may be provided to the subject via the software operating on the smartphone device 107, to the remote computerized device 105, or any other communicative protocol or system as is known in the art.

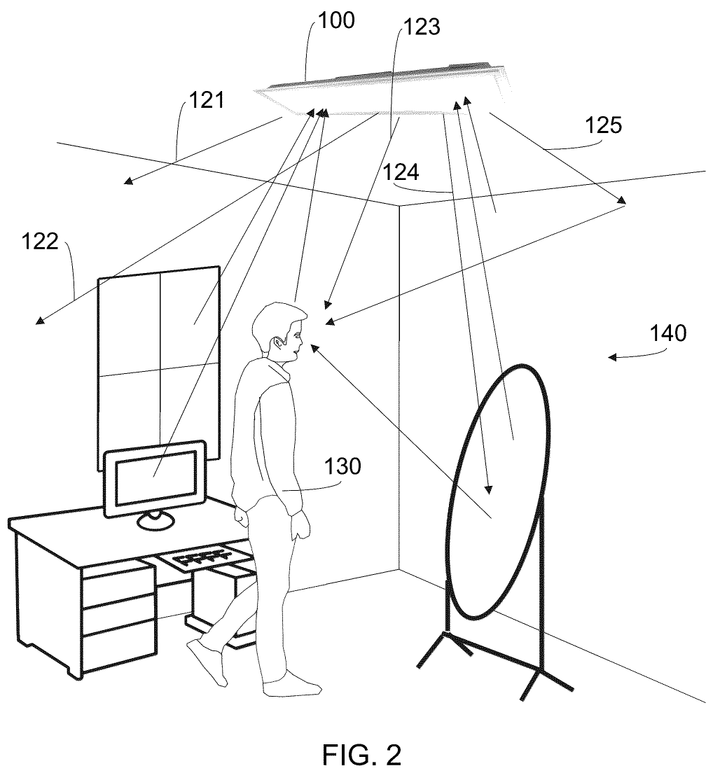

The controller 103 may control the emission characteristics of a light source 101 based on a single spatial optical characteristic or on a combination of multiple spatial optical characteristics. In embodiments in which the controller 103 controls the light source 101 based upon a distance to the target, which may be the subject, and a location of the target, which may be, by way of example and not as a limitation, the location of the target within the space or the location of the target relative to the light source, the controller 103 may calculate a horizontal intensity efficiency of therapeutic light emitted by the light source responsive to the spatial optical characteristics and operate the light source 101 further responsive to the horizontal intensity efficiency. This may optimize the amount of therapeutic light that enters the subject's retinas. Referring to FIG. 2, the system 100 may emit the therapeutic light in a variety of directions as light beams 121, 122, 123, 124, 125. As described above, the controller 103 may be configured to calculate a location of the subject 130 within a space 140. Further, the controller 103 may be configured to estimate a range within which the retinae of the subject 130 are located. Such an estimate may be based upon an estimated height of the subject 130, as may be determined by measuring the distance of the subject 130 from the system 100 and the distance of the ground of the space from the system 100. The controller 103 may further be configured to calculate the trajectory of light emitted thereby 121, 122, 123, 124, 125 and gauge whether the light is likely to directly illuminate the retinae range, such as for light beam 123, and/or be reflected by a surface in the space 140 in the direction of the retinae range, such as for light beams 124 and 125. Moreover, the controller 103 may be configured to determine light beams that are unlikely to illuminate the retinae range either directly or upon reflection, such as for light beams 121 and 122. In the present embodiment, the space 140 comprises an optically-reflective object 142. The controller 103 may be configured to determine

In one embodiment, the system 100 may include a digital communication device 104 positioned in communication with the controller 103. The digital communication device 104 may also be in communication with at least one of a remote computerized device 105, a computer network 106, or the sensors. The spatial optical characteristic may be sensed by, calculated by, provided to, or otherwise determined by the remote computerized device 105 or computer network 106 and provided to the digital communication device 104. The spatial optical characteristic may be received by the digital communication device 104 and transmitted to the controller 103.

The digital communication device 104 may be any communication device for digital communication of computers as is known in the art, including but not limited to, wired communication devices, such as Ethernet devices, Universal Serial Bus (USB) devices, and the like, and wireless communication devices, including all IEEE 802.xx compatible devices, including, but not limited to, Wi-Fi devices, Bluetooth devices, Zigbee devices, Z-Wave devices, Matter/Thread devices, and the like. Moreover, all devices operable to communicate across a computer network 106 as described above are contemplated and included within the scope of the invention. Such computer networks 106 include, but are not limited to, personal area networks, local area networks, and wide area networks, including the Internet.

By way of example, and not as a limitation, the digital communication device 104 may be positioned in communication with a smartphone device 107 or the like. the smartphone device 107 may comprise an optical sensor (not shown). The optical sensor may be adapted to capture or otherwise obtain optical measurements of the space, or environment, in which the smartphone device 107 is positioned. The smartphone device 107 may comprise software that enables the transmission of optical measurements obtained by the optical sensor of the smartphone device 107 to the digital communication device 104. Such software is contemplated as part of an embodiment the invention.

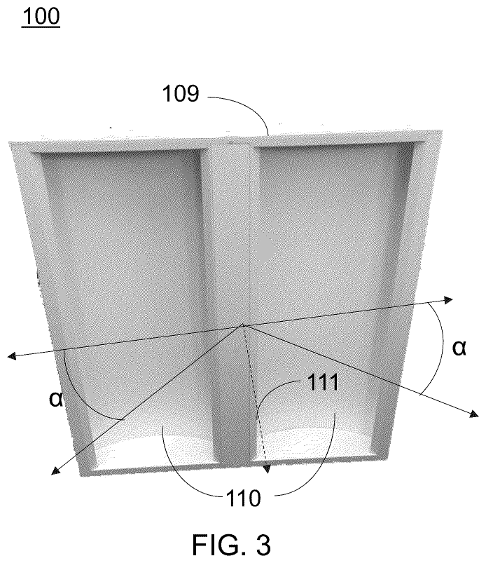

Referring now to FIG. 3, additional details of the invention are presented. The system 100 may also include a housing 109 configured to carry the light source 101. The housing 109 may also carry an optic 110. The optic 110 may be carried by and positioned within the housing 109 with respect to the light source 101 to reflect and/or refract the therapeutic light emitted by the light source 101. In some embodiments, the optic 110 may be formed by applying an optically reflective coating to a surface of the housing 109. In one embodiment, the optic may be configured to reflect and/or refract most or substantially all of the therapeutic light such that it is emitted from the housing 109 into the space at an angle α greater than 45 degrees relative to a normal of the housing 109.

In one embodiment, the housing 109 may be configured as a troffer light fixture. In such an embodiment, the optic 110 may be configured to reflect and/or refract the therapeutic light such that it is emitted into space at an angle greater than 45 degrees relative to a normal 111 of a troffer light fixture. This angle may be desirable because it may increase the amount of therapeutic light entering a subject's retinas. In an embodiment in which the therapeutic light emitted at a 45-degree angle, but is instead, by way of example, emitted directly downward toward a subject, a smaller amount of therapeutic light may enter the subject's retinas than in an embodiment in which the therapeutic light is emitted at an angle greater than 45 degrees or otherwise optimized to provide therapeutic light to the subject's retinas. When the therapeutic light is emitted at an angle, rather than straight down (e.g., parallel or close to parallel to the normal 111), the light may reflect off walls or other surrounding structures and be directed toward the subject at a height similar to the anticipated height of the user's eyes, which allows a larger percentage of the therapeutic light to enter the subject's retinas.

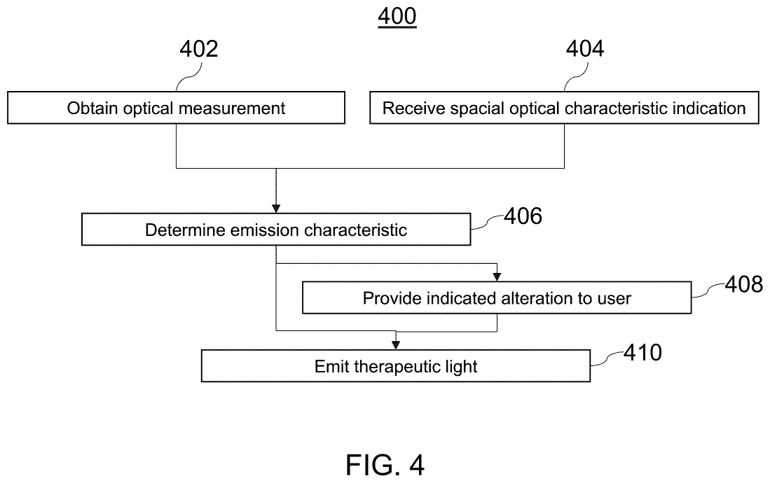

An inventive method 400 of administering therapeutic light to a subject in order to effectuate a physiological response is presented in FIG. 4. The method 400 may comprise at least one of obtaining an optical measurement of a space into which the therapeutic light may be emitted at block 402 and receiving a spatial optical characteristic indication of the space at block 404, received from, for example, a smartphone device 107 as described hereinabove. The method may further comprise determining at least one emission characteristic responsive to the spatial optical characteristic at block 406, which emission characteristic results in emitting a light within a monochromatic wavelength range that will enter the subject's retinas, defined as therapeutic light. In some embodiments, the method 400 may continue at block 408 with providing an indication to the user to alter an optical characteristic of the space responsive to the determined emission characteristic. This may include, as mentioned above, applying an optically reflective coating to a surface in the space and/or repositioning an optically reflective object within the space. The method 400 may conclude at block 410 with emitting the therapeutic light into the space.

Some of the illustrative aspects of the present invention may be advantageous in solving the problems herein described and other problems not discussed which are discoverable by a skilled artisan.

While the above description contains much specificity, these should not be construed as limitations on the scope of any embodiment, but as exemplifications of the presented embodiments thereof. Many other ramifications and variations are possible within the teachings of the various embodiments. While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best or only mode contemplated for carrying out this invention, but that the invention will include all embodiments falling within the description of the invention. Also, in the drawings and the description, there have been disclosed exemplary embodiments of the invention and, although specific terms may have been employed, they are unless otherwise stated used in a generic and descriptive sense only and not for purposes of limitation, the scope of the invention therefore not being so limited. Moreover, the use of the terms first, second, etc. do not denote any order or importance, but rather the terms first, second, etc. are used to distinguish one element from another. Furthermore, the use of the terms a, an, etc. do not denote a limitation of quantity, but rather denote the presence of at least one of the referenced item.

1. A system for selective emission of therapeutic monochromatic light comprising:
a light source operable to emit a therapeutic light within a monochromatic wavelength range, the monochromatic wavelength range being associated with a physiological response in a human subject;
a non-transitory computer-readable storage medium; and
a controller positioned in communication with each of the non-transitory computer-readable storage medium and the light source and configured to:
receive an indication of a spatial optical characteristic of a space into which the light source is positioned to emit light; and
operate the light source to emit the therapeutic light responsive to the spatial optical characteristic.

2. The system of claim 1, wherein the monochromatic wavelength range has a half-wave width of not more than 10 nm.

3. The system of claim 1, wherein the monochromatic wavelength range has a half-wave width of not more than 20 nm.

4. The system of claim 1, wherein the monochromatic wavelength range is centered at 520 nm.

5. The system of claim 1, wherein the monochromatic wavelength range is centered within a range from 510 nm to 530 nm.

6. The system of claim 1, wherein the controller is configured to operate the light source responsive to the spatial optical characteristic by controlling at least one of a peak intensity, a fluence, and a luminous flux of the therapeutic light responsive to the spatial optical characteristic.

7. The system of claim 1, wherein the spatial optical characteristic comprises at least one of a spectral power distribution of the space, a reflectivity characteristic of the space, a reflectivity model of the space, a distance to a target of the therapeutic light, a location of the target within the space, a location of the target relative to the light source, and a presence of an electronic display device in the space.

8. The system of claim 7, wherein:
the spatial optical characteristic comprises:
a distance to the target; and
at least one of a location of the target within the space and a location of the target relative to the light source; and
the controller is configured to:
calculate a horizontal intensity efficiency of therapeutic light emitted by the light source responsive to the spatial optical characteristic; and
operate the light source further responsive to the horizontal intensity efficiency.

9. The system of claim 1, further comprising a digital communication device positioned in communication with the controller and at least one of a remote computerized device and a computer network; wherein the spatial optical characteristic is received by the digital communication device and transmitted to the controller.

10. The system of claim 9, further comprising software operable to run on a smartphone device and configured to:
obtain an optical measurement of the space from an optical sensor comprised by the smartphone device;
determine the spatial optical characteristic from the optical measurement; and
transmit the spatial optical characteristic to the digital communication device utilizing a digital communication device comprised by the smartphone device.

11. The system of claim 1, further comprising an optical sensing device positioned in communication with the controller; wherein the controller is configured to:
operate the optical sensing device to obtain an obtained optical measurement of the space; and
determine the spatial optical characteristic of the space from the obtained optical measurement.

12. The system of claim 1, further comprising:
a housing configured to carry the light source; and
an optic configured to at least one of reflect and refract the therapeutic light emitted by the light source into the space.

13. The system of claim 12, wherein:
the housing, the optic, and the light source define a troffer light fixture configured to be installed in a ceiling of the space; and
the optic is configured to at least one of reflect and refract the therapeutic light such that it is emitted into the space at an angle greater than 45 degrees relative to a normal of the troffer light fixture.

14. A method of administering therapeutic light to a subject to effectuate a physiological response comprising:
receiving an indication of a spatial optical characteristic of a space into which a light source is positioned to emit light; and
determining at least one emission characteristic responsive to the spatial optical characteristic to emit light within a monochromatic wavelength range, defining a therapeutic light, the monochromatic wavelength range being associated with a physiological response in a human subject; and
emitting the therapeutic light into the space.

15. The method of claim 14, wherein the monochromatic wavelength range has a half-wave width of not more than 10 nm.

16. The method of claim 14, wherein the monochromatic wavelength range is centered at 520 nm.

17. The method of claim 14, wherein the spatial optical characteristic comprises at least one of a spectral power distribution of the space, a reflectivity characteristic of the space, a reflectivity model of the space, a distance to a target of the therapeutic light, and a presence of an electronic display device in the space.

18. The method of claim 14, wherein the spatial optical characteristic is received from at least one of a directly connected computerized device and a computerized device connected across a computerized network.

19. The method of claim 14, further comprising:
obtaining an obtained optical measurement of the space; and
determining the spatial optical characteristic of the space from the obtained optical measurement.

20. The method of claim 14, further comprising providing an indication to a user to alter the spatial optical characteristic, defining an indicated alteration.

21. The method of claim 20, wherein the indicated alteration to the spatial optical characteristic comprises at least one of applying an optically-reflective coating to a surface within the space and positioning an optically-reflective object within the space.

22. The method of claim 14, wherein the emission characteristic is at least one of a peak intensity, a fluence, and a luminous flux of the therapeutic light.
